(19) United States
(12) Patent Application Publication
(10) Pub. No.: US 2026/0207387 A1
(43) Pub. Date: Jul. 23, 2026

(54) ASTIGMATISM PERSONAL PROTECTIVE GEAR DEVICE

(71) Applicant: Ryan Schaper, Auburn, IN (US)

(72) Inventor: Ryan Schaper, Auburn, IN (US)

(21) Appl. No.: 19/243,996

(22) Filed: Jun. 20, 2025

Related U.S. Application Data
(60) Provisional application No. 63/747,515, filed on Jan. 21, 2025.

(51) Int. Cl. A61F 9/04

(52) U.S. Cl. CPC A61F 9/045

(57) ABSTRACT

A specialized eyewear device is disclosed which comprises a goggle component that is configured in the base shape of a traditional pair of goggles or glasses. Two lenses are used to cover the ocular sockets of the user and are connected to an adjustable elastic strap which fits around the user's head. Further, the goggle style ensures that the eyes remain free of pressure during sleep while also maintaining comfort for the user. Thus, the goggle component is relatively small, comfortable, and adjustable via the adjustable elastic strap.

### CROSS-REFERENCE TO RELATED APPLICATION

The present application claims priority to, and the benefit of, U.S. Provisional Application No. 63/747,515, which was filed on Jan. 21, 2025, and is incorporated herein by reference in its entirety.

### FIELD OF THE INVENTION

The present invention relates generally to the field of specialized eyewear devices. More specifically, the present invention relates to a specialized pair of eyewear designed to protect the eyes from pressure during sleep. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in specialized eyewear devices. Generally, pressure applied to the eyes during sleep can lead to the development or recurrence of astigmatism. People who recently underwent Lasik surgery may be at a high risk of developing astigmatism due to contact with pillows and bed surfaces while asleep. Accordingly, recovery after Lasik is important. Further, the corrective surgery only remains successful if the recovery is successful.

Accordingly, eyewear constructed in accordance with the invention can be easily applied to the eyes to ensure that there is minimal to no pressure during sleep. Further, it is often necessary for people to sleep with their eyes in contact with the bed. These sleep positions increase the chance of astigmatism developing without the proper eyewear. Accordingly, certain eyewear is typically used, which can be uncomfortable for the user and overall cause less sleep and recovery for the eye.

Accordingly, there is a demand for an improved specialized eyewear device that allows users to prevent the development or recurrence of astigmatism. More particularly, there is a demand for a specialized eyewear device that ensures the eyes remain free of pressure during sleep.

Therefore, there exists a long-felt need in the art for a specialized eyewear device that provides users with flexible and comfortable goggle-style eyewear that protects eyes from development or recurrence of astigmatism. There is also a long-felt need in the art for a specialized eyewear device that features a low-profile configuration with a padded frame and adjustable elastic strap for comfort. Further, there is a long-felt need in the art for a specialized eyewear device that includes clear, smoke, or blackout color lenses to accommodate people who prefer transparent or complete darkness while resting. Moreover, there is a long-felt need in the art for a device that helps improve recovery after corrective eye surgeries like Lasik to prevent any pressure on the eyes from causing injury or impacting the surgery. Further, there is a long-felt need in the art for a specialized eyewear device that provides a more comfortable way to sleep with eyewear on. Finally, there is a long-felt need in the art for a specialized eyewear device that includes various different sizes and colors to accommodate for user specifications.

The subject matter disclosed and claimed herein, in one embodiment thereof, comprises a specialized eyewear device. The device is a modified pair of eyewear designed to protect the eyes from pressure during sleep. The specialized eyewear device comprises a goggle component that is configured in the base shape of a traditional pair of goggles or glasses. Two lenses are used to cover the ocular sockets of the user and are connected to an adjustable elastic strap which fits around the user's head. Further, the goggle style ensures that the eyes remain free of pressure during sleep while also maintaining comfort for the user. Thus, the goggle component is relatively small, comfortable, and adjustable via the adjustable elastic strap. The lenses can come in various appearances, such as clear, smoke, and blackout, to also provide comfort for the user during sleep. Further, the goggle component comprises multiple different sizes and colors, which can change based on the preferences of the user.

In this manner, the specialized eyewear device of the present invention accomplishes all of the foregoing objectives and provides users with a device that provides a specialized pair of eyewear. The device is a pair of eyewear which prevents pressure from building on the eyes during sleep. The device can comprise a plurality of sizes.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a specialized eyewear device. The device is a modified pair of eyewear designed to protect the eyes from pressure during sleep. The specialized eyewear device comprises a goggle component that is configured in the base shape of a traditional pair of goggles or glasses. Further, the goggle style ensures that the eyes remain free of pressure during sleep while also maintaining comfort for the user. Thus, the goggle component is relatively small, comfortable, and adjustable via the adjustable elastic strap. Further, the goggle component comprises multiple different sizes and colors, which can change based on the preferences of the user.

In one embodiment, the specialized eyewear device allows users to sleep comfortably without causing pressure to build on the eyes. Further, the adjustable elastic strap and padded frame allow for comfort for the user. Accordingly, users can sleep with the eyewear on to prevent pressure on the eyes from contact with pillows or bed sheets, or to protect the eyes while the user is recovering from vision correction surgery such as Lasik.

In one embodiment, the specialized eyewear device comprises a goggle component that is configured in a traditional goggle or glasses base shape. The goggle component comprises an exterior surface, an interior surface, and a side edge along the circumference of the goggle component. However, the goggle component can be any suitable shape and size as is known in the art, as long as the goggle component is sized and shaped to be worn over the eyes of the user.

In one embodiment, the goggle component comprises a lens feature which encompasses most of the goggle component. The lens feature is comprised of a glass material and completely covers the ocular socket of the eye. There are two sections of the lens feature for each of the user's eyes, with a connection of the same glass material over the bridge of the user's nose.

In one embodiment, the lens feature is comprised of multiple different colors, such as clear, smoke, or blackout. These multiple different colors allow for the user to be more comfortable while sleeping, since they allow for the device to be used as a sleep mask as well.

In one embodiment, the goggle component comprises a padded frame surrounding the side edge of the goggle component. The padded frame is slightly raised from the lens feature it surrounds and is comprised of a comfortable material, such as foam or fleece. The padded frame allows for comfort for the user during sleep.

In one embodiment, the goggle component also comprises an adjustable elastic strap. This adjustable elastic strap connects to the padded frame on either side of the device. The adjustable elastic strap is large enough to fit around the user's head, where it can then be adjusted to the tightness preferred by the user.

In one embodiment, the specialized eyewear device can be any suitable size to accommodate for user preferences. The device can also be any suitable color along the adjustable elastic strap and the padded frame, as is suitable to the user.

In one embodiment, the specialized eyewear device is manufactured from glass, plastic, or other suitable materials as is known in the art, or a combination of such materials.

In yet another embodiment, the specialized eyewear device comprises a plurality of indicia.

In yet another embodiment, a method of protecting the eyes during sleep is disclosed. The method includes the steps of providing a specialized eyewear device comprising a goggle component with a lens feature and an adjustable elastic strap. The method also comprises adjusting the length of the adjustable elastic strap to be comfortable for the user. Further, the method comprises stretching the adjustable elastic strap over the user's head. The method also comprises adjusting the goggle component so that the lens feature fits over the ocular sockets. Finally, the method comprises sleeping without the worry of added pressure on the eyes, causing vision damage.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the specialized eyewear device of the present invention showing the device in accordance with the disclosed architecture;

FIG. 2 illustrates a perspective view of one embodiment of the specialized eyewear device of the present invention showing the padded frame in accordance with the disclosed architecture;

FIG. 3 illustrates a perspective view of one embodiment of the specialized eyewear device of the present invention showing the adjustable elastic strap in accordance with the disclosed architecture;

FIG. 4 illustrates a perspective view of one embodiment of the specialized eyewear device of the present invention showing the various sizes in accordance with the disclosed architecture;

FIG. 5 illustrates a perspective view of one embodiment of the specialized eyewear device of the present invention showing the device in use in accordance with the disclosed architecture; and

FIG. 6 illustrates a flowchart showing the method of protecting the eyes during sleep in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a specialized eyewear device that provides users with flexible and comfortable goggle-style eyewear that protects eyes from development or recurrence of astigmatism. There is also a long-felt need in the art for a specialized eyewear device that features a low-profile configuration with a padded frame and adjustable elastic strap for comfort. Further, there is a long-felt need in the art for a specialized eyewear device that includes clear, smoke, or blackout color lenses to accommodate people who prefer transparent or complete darkness while resting. Moreover, there is a long-felt need in the art for a device that helps improve recovery after corrective eye surgeries like Lasik to prevent any pressure on the eyes from causing injury or impacting the surgery. Further, there is a long-felt need in the art for a specialized eyewear device that provides a more comfortable way to sleep with eyewear on. Finally, there is a long-felt need in the art for a specialized eyewear device that includes various different sizes and colors to accommodate for user specifications.

The present invention, in one exemplary embodiment, is a novel specialized eyewear device. The specialized eyewear device comprises a goggle component that is configured in the base shape of a traditional pair of goggles or glasses. Two lenses are used to cover the ocular sockets of the user and are connected to an adjustable elastic strap which fits around the user's head. Further, the goggle style ensures that the eyes remain free of pressure during sleep while also maintaining comfort for the user. The lenses can come in various appearances, such as clear, smoke, and blackout, to also provide comfort for the user during sleep. Further, the goggle component comprises multiple different sizes and colors, which can change based on the preferences of the user. The present invention also includes a novel method of protecting the eyes during sleep. The method includes the steps of providing a specialized eyewear device comprising a goggle component with a lens feature and adjustable elastic strap. The method also comprises adjusting the length of the adjustable elastic strap to be comfortable for the user. Further, the method comprises stretching the adjustable elastic strap over the user's head. The method also comprises adjusting the goggle component so that the lens feature fits over the ocular sockets. Finally, the method comprises sleeping without the worry of added pressure on the eyes causing vision damage.

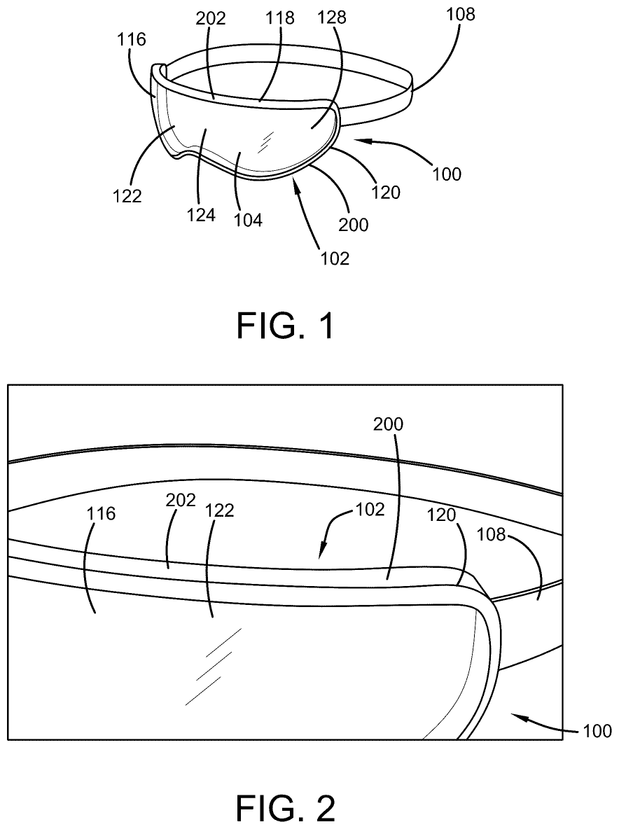

Referring initially to the drawings, FIG. 1 illustrates a perspective view of one embodiment of the specialized eyewear device 100 of the present invention. In the present embodiment, the specialized eyewear device 100 is an improved specialized eyewear device 100 that provides a user 106 with goggles which protect the eyes and are comfortable to sleep in. Specifically, the specialized eyewear device 100 comprises a goggle component 102 with a lens feature 104. Thus, the goggle component 102 is relatively small, comfortable, and adjustable via an adjustable elastic strap 108. Further, the lens feature 104 and the goggle component 102 can be various sizes depending on the preference of the user 106.

Generally, the specialized eyewear device 100 allows users 106 to protect their eyes 110 from astigmatism, which can occur during sleep because of pressure placed on the eye 110. Further, the goggle component 102 can be used to relieve pressure that is placed on the eye 110 from contact with pillows 112 or bed sheets 114. Accordingly, users 106 can manipulate the goggle component 102 (i.e., moving, adjusting, tightening, loosening, etc.), solely via the adjustable elastic strap 108, preventing pain and discomfort.

Further, the specialized eyewear device 100 comprises a goggle component 102 that is configured in a traditional goggle or glasses base shape. The goggle component 102 comprises an exterior surface 116, an interior surface 118, and a side edge 120 along the circumference of the goggle component 102. However, the goggle component 102 can be any suitable shape and size as is known in the art, as long as the goggle component 102 is sized and shaped to be worn over the eyes 110 of the user 106.

Additionally, the goggle component 102 comprises a low-profile configuration with a lens feature 104, which encompasses most of the goggle component 102. The lens feature 104 is comprised of a glass material 122 and completely covers the ocular socket of the eye 110. There are two sections of the lens feature 104 for each of the user's eyes 110, with a connection 124 of the same glass material 122 over the bridge of the user's nose 126.

In one specific embodiment, the lens feature 104 is comprised of multiple different colors 128 such as clear, smoke, or blackout. These multiple different colors 128 allow for the user 106 to be more comfortable while sleeping, since they allow for the device 100 to be used as a sleep mask as well.

As shown in FIG. 2, the goggle component 102 comprises a padded frame 200 surrounding the side edge 120 of the goggle component 102. The padded frame 200 is slightly raised from the lens feature 104 it surrounds and is comprised of a comfortable material 202 such as foam or fleece. The padded frame 200 allows for comfort for the user 106 during sleep and for there to be no pressure on the eye 110.

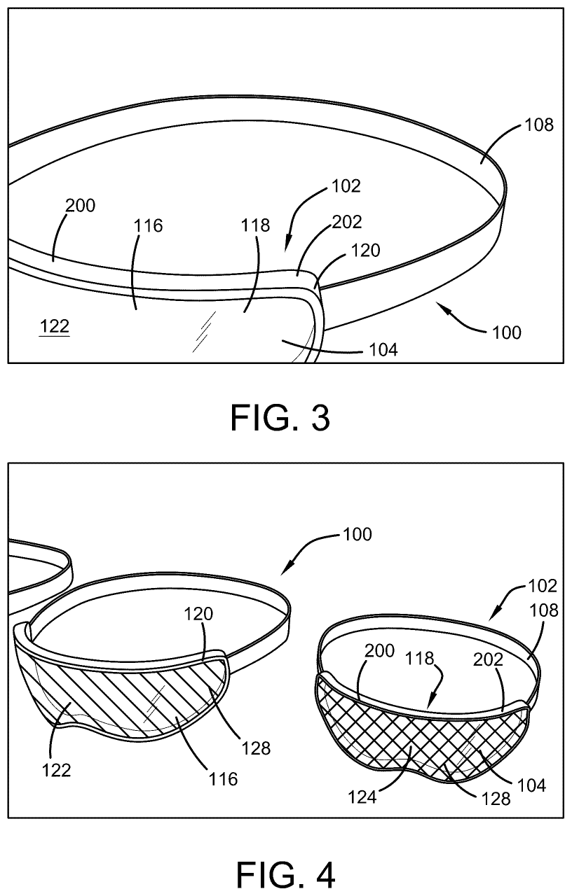

As shown in FIG. 3, the goggle component 102 also comprises an adjustable elastic strap 108. This adjustable elastic strap 108 connects to the padded frame 200 on either side of the device 100. The adjustable elastic strap 108 is large enough to fit around the user's head 300 where it can then be adjusted to the tightness preferred by the user 106

As shown in FIG. 4, the specialized eyewear device 100 can be any suitable size to accommodate for user 106 preferences. The specialized eyewear device 100 can also be any suitable color along the adjustable elastic strap 108 and the padded frame 200 as is suitable to the user 106.

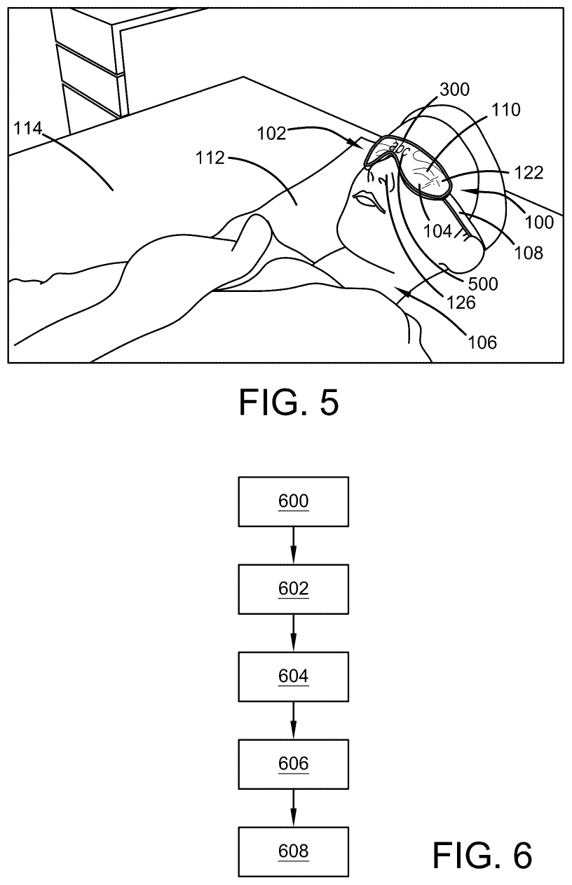

As shown in FIG. 5, the specialized eyewear device 100 is manufactured from glass, plastic, or other suitable materials 122 as is known in the art, or a combination of such materials 122.

In yet another embodiment, the specialized eyewear device 100 comprises a plurality of indicia 500. The goggle component 102 of the device 100 may include advertising, a trademark, or other letters, designs, or characters, printed, painted, stamped, or integrated into the goggle component 102, or any other indicia 500 as is known in the art. Specifically, any suitable indicia 500 as is known in the art can be included, such as, but not limited to, patterns, logos, emblems, images, symbols, designs, letters, words, characters, animals, advertisements, brands, etc., that may or may not be goggle, eye, or brand related.

FIG. 6 illustrates a flowchart of the method of protecting the eyes during sleep. The method includes the steps of at 600, providing a specialized eyewear device comprising a goggle component with a lens feature and an adjustable elastic strap. The method also comprises at 602, adjusting the length of the adjustable elastic strap to be comfortable for the user. Further, the method comprises at 604, stretching the adjustable elastic strap over the user's head. The method also comprises at 606, adjusting the goggle component so that the lens feature fits over the ocular sockets. Finally, the method comprises at 608, sleeping without the worry of added pressure on the eyes causing vision damage.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “specialized eyewear device”, “eyewear device”, “goggle device”, and “device” are interchangeable and refer to the specialized eyewear device 100 of the present invention.

Notwithstanding the foregoing, the specialized eyewear device 100 of the present invention can be of any suitable size and configuration as is known in the art without affecting the overall concept of the invention, provided that it accomplishes the above-stated objectives. One of ordinary skill in the art will appreciate that the specialized eyewear device 100 as shown in FIGS. 1-6 is for illustrative purposes only, and that many other sizes and shapes of the specialized eyewear device 100 are well within the scope of the present disclosure. Although the dimensions of the specialized eyewear device 100 are important design parameters for user convenience, the specialized eyewear device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A specialized eyewear device that provides a user with a pair of eyewear to prevent pressure from building on the eye during sleep, the specialized eyewear device comprising:
a goggle component; and
a lens feature;
wherein the lens feature encompasses most of the goggle component and covers ocular sockets of an eye of a user; and
further wherein the lens feature is attached to an adjustable elastic strap.

2. The specialized eyewear device of claim 1, wherein the goggle component is configured in a base shape of a traditional pair of goggles with a lens, frame, and strap.

3. The specialized eyewear device of claim 2, wherein the goggle component comprises an exterior surface, an interior surface, and a side edge along a circumference of the goggle component.

4. The specialized eyewear device of claim 3, wherein the goggle component comprises a padded frame surrounding the side edge which is slightly raised compared to the lens feature.

5. The specialized eyewear device of claim 4, wherein the padded frame is comprised of a foam or fleece material or any such material that would be comfortable to a user during sleep.

6. The specialized eyewear device of claim 5, wherein the goggle component comprises an adjustable elastic strap which is connected on either side of the google component and fits around a user's head.

7. The specialized eyewear device of claim 6, wherein the goggle component and adjustable elastic strap can come in various sizes and colors to accommodate for user's preferences.

8. The specialized eyewear device of claim 7, wherein the lens feature may be comprised of multiple different colors as well, including clear, smoke, and blackout.

9. The specialized eyewear device of claim 8, wherein the lens feature of the goggle component is comprised of a glass material, plastic material, or a combination of such materials.

10. The specialized eyewear device of claim 9, wherein the adjustable elastic strap allows the user to tighten or loosen a fit of the goggle component to their preferences.

11. The specialized eyewear device of claim 10, wherein the goggle component can be specifically used for patients recovering from vision reconstruction, such as surgery.

12. The specialized eyewear device of claim 11 further comprising a plurality of indicia.

13. A specialized eyewear device that provides a user with a pair of eyewear which relieves pressure from a user's eyes while sleeping, the specialized eyewear device comprising:
a goggle component configured in a base shape of a traditional pair of goggles; and
a lens feature;
wherein the goggle component comprises an exterior surface, an interior surface, and a side edge along a circumference of the goggle component;
wherein the goggle component can be loosened or tightened to a user by an adjustable elastic strap;
wherein the lens feature completely covers ocular sockets of an eye and connects over a bridge of a user's nose;
wherein the lens feature is outlined by a padded frame which lines the side edge of the goggle component; and
further wherein the adjustable elastic strap connects to either side of the goggle component and fits around a user's head.

14. The specialized eyewear device of claim 13 further comprising a plurality of indicia.

15. The specialized eyewear device of claim 13, wherein the lens feature is comprised of multiple different colors including clear, smoke, and blackout in order to accommodate for different sleeping preferences.

16. The specialized eyewear device of claim 15, wherein the lens feature is comprised of a glass material, plastic material, or any combination of such materials.

17. The specialized eyewear device of claim 16, wherein the padded frame is comprised of a fleece or foam material and is slightly raised compared to the lens feature of the goggle component.

18. The specialized eyewear device of claim 17 wherein the goggle component can be comprised of various sizes or colors to accommodate for user preferences.

19. The specialized eyewear device of claim 18 wherein the device is comprised of a low-profile configuration.

20. A method of protecting the eyes during sleep, the method comprising the following steps:
providing a specialized eyewear device comprising a goggle component with a lens feature and adjustable elastic strap;
adjusting the length of the adjustable elastic strap to be comfortable for the user;
stretching the adjustable elastic strap over the user's head;
adjusting the goggle component so that the lens feature fits over the ocular sockets; and
sleeping without the worry of added pressure on the eyes causing vision damage.
